(19) United States
(12) Patent Application Publication
(10) Pub. No.: US 2007/0286848 A1
(43) Pub. Date: Dec. 13, 2007

(54) Epas1 Gene Transfer to Improve Cell Therapy

(76) Inventors: Guy Louis-Georges, Montreal (CA); Fortin Anouk, Gloucester (CA)

Correspondence Address: SIM & MCBURNEY, 330 UNIVERSITY AVENUE, 6TH FLOOR, TORONTO, ON, M5G 1R7, CA

(21) Appl. No.: 10/579,679

(22) PCT Filed: Jun. 4, 2004
(86) PCT No.: PCT/CA04/00837

§ 371 Date: Feb. 5, 2007

Related U.S. Application Data
(60) Provisional application No. 60/476,624, filed on Jun. 9, 2003.

(30) Foreign Application Priority Data
Jun. 5, 2003 (CA) 2,431,425

(51) Int. Cl. A61K 48/00; A01K 67/00; A61P 17/02; A61P 9/00; C12N 5/06; C12N 5/10

(52) U.S. Cl. 424/93.21; 435/366; 435/371; 435/372; 435/455; 435/461; 800/13

(57) ABSTRACT

The present invention concerns the use of nucleotide sequences encoding EPAS1, HIF-1α and HIF-3α transcription factors and functional analogs for treating coronary and cardiac diseases in mammals. The use of such transcription factors and its analogs is useful in the treatment of disorders that may be treated by cell therapy such as peripheral vascular disease (PVD), neurodegenerative disease including Parkinson's syndrome, muscular dystrophies, stroke, diabetes, hemophilia, wound and others.

### BACKGROUND OF THE INVENTION

a) Field of the Invention

The present invention relates to methods and composition of matter for improving cell implantation and cardiac function.

b) Description of the Prior Art

Chronic ischemic heart disease is a worldwide health problem of major proportions. According to the American Heart Association, 61 800 000 Americans have at least one type of cardiovascular disease⁽¹⁾. In particular, coronary heart disease (CHD) cause myocardial infarction (MI) for 7 500 000 American patients and congestive heart failure (CHF) for 4 800 000 American patients. Almost 450 000 deaths in the United States alone were deemed to derive from CHD⁽¹⁾.

Current CHD treatments include medication, percutaneous transluminal coronary angioplasty and coronary artery bypass surgery. These procedures are quite successful to increase blood flow in the myocardium thus reducing ischemia and ameliorating the condition of the patient. However, due to the progressive nature of CHD, the beneficial effects of these procedures are not permanent and new obstructions can occur. Patients that live longer through effective cardiovascular interventions eventually run out of treatment options. Also an important patient population is still refractory to these treatments due to diffuse athereosclerotic diseases and/or small caliber arteries.

Severe and chronic ischemia can cause MI which is an irreversible scarring of the myocardium. This scarring reduces heart contractility and elasticity and consequently the pumping function, which can then lead to CHF. Treatments available to CHF patients target kidney function and peripheral vasculature to reduce the symptoms but none are treating the scar or increasing pump function of the heart.

An emerging treatment for CHF patients is cellular cardiomyoplasty (CCM), a treatment aiming at reducing the scar and improving heart function. It consists in the injection of cells in the scar, replacing the fibrotic scar by healthy tissue and increasing elasticity. When the injected cells are of muscular origin, they can also contribute to contractility. The net result of this cell therapy is an improvement in heart function. Coupling CCM with therapeutic angiogenesis can improve engraftment of injected cells by increasing the blood supply to the injected cells. Furthermore, the adjacent tissue will benefit from the relief of ischemia. An important limitation of CCM is the high cell death rate at the early stages after implantation. It would be highly desirable to improve cell survival in order to increase efficacy of the treatment.

Regulators of hypoxia include the transcription factors of the Hypoxia Inducible Factors family (HIF). These include HIF-1α (also known as MOP1²; and are discussed in U.S. Pat. Nos. 5,882,314; 6,020,462 and 6,124,131, Endothelial PAS 1 (EPAS1), (also known as HIF-2α, MOP2, HIF-related factor (HRF) and HLF (HIF-like factor)³, and are also discussed in U.S. Pat. No. 5,695,963, and the newly discovered HIF-3α⁴.

These factors are highly labile in normal conditions, but are stabilized in response to low oxygen tension. This stabilization allows them to bind to cis DNA elements of target genes, and stimulate transcription of hypoxia induced genes that help cell survival in low oxygen conditions. These target genes are implicated in processes such as anaerobic metabolism (glucose transporters and glycolytic enzymes), vasodilatation (inducible nitric oxide synthase (iNOS) and heme oxygenase-1 (HO-1)), increased breathing (tyrosine hydroxylase), erythropoiesis (erythropoietin) and angiogenesis (VEGF).

However, prior to the present invention, it has never been demonstrated or suggested that EPAS1, HIF-1α and HIF-3α could induce the expression of cell induced cell survival genes, nor that EPAS1, HIF-1α and HIF-3α modified cell transplanted increased cell survival in vivo as indicated by increased metabolic activity in the cells they are introduced in. Among the cell survival genes some improve cell survival, for instance, by inhibiting apoptosis and others have a cardioprotective activity, preventing scarring of the heart tissue and reducing heart failure.

### SUMMARY OF THE INVENTION

An object of the present invention is to provide a method and compositions of matter for improving cell therapy treatment by increasing cell survival.

Another object of the invention is to provide a method and compositions of matter for improving cardioprotection, which prevents myocardial scarring and reduces heart failure.

More particularly, the present invention is concerned with the use of nucleotide sequences encoding EPAS1, HIF-1α and HIF-3α transcription factors and functional analogs for treating coronary and cardiac diseases in mammals. The use of such transcription factors and its analogs may also be useful in the treatment of disorders that may be treated by cell therapy such as peripheral vascular disease (PVD), neurodegenerative disease including Parkinson's syndrome, muscular dystrophies, stroke, diabetes, hemophilia, wound and others.

An advantage of the present invention is that it provides more effective means for inducing the expression of a plurality of cell survival genes and thereby stimulating cell survival.

The invention is thus very useful for the treatment of coronary and cardiac diseases in mammals and more particularly for the relief of myocardial ischemia, the regeneration of cardiac tissue subsequent to a myocardial infarction and for the reduction of CHD and also in peripheral vascular disease (PVD).

Tissue engineering constructs, such as skin equivalent to treat skin ulcers, would benefit from an EPAS1, HIF-1α and HIF-3α treatment.

Other objects and advantages of the present invention will be apparent upon reading the following non-restrictive description of several preferred embodiments, made with reference to the accompanying drawings.

### BRIEF DESCRIPTION OF THE DRAWINGS

FIG. 1 is a bar graph indicating the change in metabolic activity in a scarred area of rat hearts following treatment with autologous myoblasts modified or not with EPAS1 gene. High metabolic activity indicate a high cell survival and prevention of scarring.

### DETAILED DESCRIPTION OF THE INVENTION

A) General Overview of the Invention

An object of the invention is to provide methods and cells for improving cell therapy treatment such as CCM by increasing cell survival. The methods of the present invention are particularly useful for treating coronary and cardiac diseases in mammals. The invention also provides genetically modified cells expressing a plurality of cell survival genes.

The invention is based on the use of a nucleotide sequence encoding a functional EPAS1, HIF-1α or HIF-3α transcription factor or a functional analog thereof for improving cellular survival in engraftment procedures, cell therapy and/or coronary and cardiac treatments and for improving the metabolic activity of a muscular cell.

As it will be shown in the exemplification section, the present inventors have demonstrated that the induction of expression of EPAS1, HIF-1α and HIF-3α transcription factors stimulate the expression of cell survival genes such as leukemia inhibitory factor (LIF), leukemia inhibitory factor receptors (LIF-R), cardiotrophin 1 (CT 1) and adrenomedullin in myoblasts which in turn increases cell survival. The inventors showed, in a rat model of CHF, that EPAS1 modified cells transplanted in the scar tissue survived better and improved metabolic activity. It is expected that these genes are also stimulated by EPAS1, HIF-1α and HIF-3α in other cell types.

In the context of the present invention, the expression “cardioprotective gene” refers to a gene that can prevent the formation of myocardial scar and heart failure following a myocardial infarction.

The expression “cell survival gene” refers to a gene that can prevent cell death in stress condition, such as high hypoxia or implantation in a new host milieu.

B) Methods of Treatment

According to a first aspect, the invention is directed to a method for improving cell therapy by increasing cell survival and cardioprotection by inducing in a cell such as a muscular mammalian cell, the expression of at least one cell survival gene. The method comprises the step of introducing and expressing in the cell a nucleic acid sequence encoding a functional EPAS1, HIF-1α and HIF-3α transcription factor or a functional analog thereof.

In a further aspect, the invention is directed to a method for improving cardiac tissue functions of a mammal, comprising the step of providing to the cardiac tissue of the mammal a plurality of genetically modified cells expressing a nucleic acid sequence encoding a functional EPAS1, HIF-1α and HIF-3α transcription factor or a functional analog thereof.

The inventors have found that EPAS1 gene transfer induces the expression of a plurality of cell survival genes such as LIF, LIF-R, adrenomedullin and cardiotrophin 1.

HIF-1α is described in Wang et al. Proc. Natl. Aca. Sci. (1995) 92:5510-5514 and in U.S. Pat. Nos. 5,882,314; 6,020,462 and 6,124,131. EPAS1 is described in Tian et al. Genes & Dev. (1996) 11:72-82 and U.S. Pat. No. 5,692,963. HIF-3α is described in Gu et al. Gene Expression (1998) 7:205-213 and U.S. provisional application 60/292,630 filed on May 22, 2001. All these documents are incorporated herein by reference.

According to a preferred embodiment, the nucleic acid sequence encoding the transcription factor used in the present invention is a cDNA. The nucleotide sequence may be introduced in the cell or tissue using well known methods. Indeed, the sequence(s) may be introduced directly in the cells of a given tissue, injected in the tissue, or introduced via the transplantation of previously genetically modified compatible cells. For instance, this may be achieved with adenoviral vectors, plasmid DNA transfer (naked DNA or complexed with liposomes) or electroporation. Methods for introducing a nucleotide sequence into eukaryote cells such as mammalian muscular cells or for genetically modifying such cells are well known in the art. Isner Nature (2002) 415:234-239 discusses myocardial gene therapy methods and US patent application US20010041679A1 or U.S. Pat. No. 5,792,453 provides methods of gene transfer-mediated angiogenesis therapy.

In a preferred embodiment, a plurality of genetically modified cells are transplanted into the heart of a compatible recipient. In this embodiment, the transplantation is autologous. The transplantation improves the survival of implanted cells. Transplantation methods are well known in the art. For detailed examples of muscular cell transplantation, one may refer to U.S. Pat. Nos. 5,602,301 and 6,099,832.

In another preferred embodiment, the muscle cell or the muscular tissue is an ischemic muscular tissue. Accordingly, the expression of at least one cell survival gene and/or the transplantation of previously genetically modified compatible cells in these ischemic cells or tissue increases tissue function. Also, the efficacy of cell survival and engraftment being a limiting step, the expression of at least one cell survival gene is desirable.

It should be noted that in both of these preferred embodiments, the level of expression of the transcription factor(s) is such that the cell survival genes are expressed at a level that is sufficient to improve cell survival and sustain cardioprotection. For a better control on the expression and selectivity of these cell survival genes, the transcription factor may be inducible.

In a further aspect, the invention is directed to a genetically modified cell expressing a functional EPAS1, HIF-1α and HIF-3α transcription factor or a functional analog thereof. Preferably also, the cell comprises a cDNA encoding the transcription factor. Preferably, the cell is a myoblast, a skeletal muscular cell or a cardiac cell. The genetically modified cells could also be components of bone marrow, fibroblasts or stem cells. The nature of the cell used in the methods of the present invention will vary depending on the disorder to be treated. In conditions such as dystrophies, cells such as myoblasts are useful. In stroke and Parkinson's disease, neurons or bone marrow cells may be useful and in diabetes, pancreatic islets cells may be useful. For the treatment of wounds, fibroblasts or keratinocytes are useful.

As mentioned previously, such cells may be particularly useful when transplanted in a compatible recipient for increasing the metabolic activity of a mammalian muscular tissue, and/or increasing muscular function in CHF, locally or in surrounding transplanted tissue.

Of course, the genetically modified cells of the present invention could also be used for the formation of artificial organs or for tissue constructions. Also, other cell types, such as bone marrow cells and their sub-populations, fibroblasts, smooth muscle cells, endothelial cells, endothelial progenitor cells and embryonic stem cells, have other desirable properties for the implantation in other tissue or other type of muscle. Genetic modification of these cells with EPAS1, HIF-1α and HIF-3α to improve perfusion and engraftment is also an aspect of the invention.

As it will now be demonstrated by way of an example hereinafter, the present invention is useful for increasing cell survival and tissue function in CHD and in PVD.

### EXAMPLES

The following example is illustrative of the wide range of applicability of the present invention and is not intended to limit its scope. Modifications and variations can be made therein without departing from the spirit and scope of the invention. Although any method and material similar or equivalent to those described herein can be used in the practice for testing of the present invention, the preferred methods and materials are described.

### Example 1

### Use of EPAS1 to Induce Angiogenesis

1) Materiel and Methods

Adenovirus Production

EPAS1/pcDNA3 plasmid was kindly provided by S. L. McKnight⁽³⁾ and was used to produce adenoviral vectors with the Ad.Easy™ technology using manufacturer methodology (Q-Biogene).

Infection

Early passage human (Clonetics) or rat myoblasts were plated in 100 mm dishes and grown until they reached ˜70% confluence. Cells were rinsed with PBS and covered with 4 ml DMEM with 10% fetal calf serum (FCS) and adenoviruses at a MOI of 500. Cells were incubated at 37° C. with constant but gentle agitation for 6 hours. 6 ml of DMEM with 10% FCS was added and cells were incubated overnight at 37° C.

Gene Chip Hybridization

Total RNA was isolated from human myoblasts (Clonetics) infected with either Ad.Null™ (Q-Biogene) or Ad.EPAS1 as described⁽⁷⁾. Probes were prepared and hybridized to Atlas Human 1.2 Array (Clontech) and to 8K Human Atlas Array (Clontech) according to the manufacturer's instructions. The arrays were exposed to phosphorimager screen and analyzed with the Atlas 2.01 software (Clontech).

Cell Survival in Infarct Heart

Normal or EPAS1 modified rat autologous myoblasts were implanted in infarcted rat hearts 10 days after permanent left anterior descending coronary artery ligation (Myoinfarct™ rats, Charles River Laboratories) by direct myocardial injection of 2 millions cells via a mini-thoracotomy (N=12). Metabolic activity was measured 5 days post ligature and 8 weeks post treatment by injection of ¹⁸FDG acquisition using a small animal PET-Scan (Sherbrooke University). FDG uptake in the infarct was quantified and a % change (post vs pre treatment) was calculated.

2) Results

Activation of Cell Survival Genes by EPAS1 In Vitro

To evaluate EPAS1 potential as a cell survival modulator, gene expression was compared in human Myoblast infected either with Ad.EPAS1 or Ad.Null™ using gene chip technology. cDNA probes derived from either cell population was hybridized on a Atlas human 1.2 Array™ or 8K Human Atlas Array (Clontech) assessing expression of almost 1200 genes or 8000 genes. Cell survival and cardioprotective genes were also found to be upregulated by EPAS1: LIF is known to enhance survival of Myoblast, which would be useful in cell therapy. Its receptor, LIF-R, was also stimulated. In the same gene family, cardiotrophin 1 (CT-1) enhances muscle cells survival and protects from heart injury. CT-1 is a survival factor for cardiomyocytes. Adrenomedullin is a potent cardioprotective gene, it has a beneficial effect on left ventricular remodeling after MI and helps prevent heart failure.

TABLE 1
Genes activated by EPAS1.

| Gene | Fold induction | Category |
|---|---|---|
| LIF | up | Growth factor |
| LIF-R | up | Receptor |
| Adrenomedullin | 4.87 | Growth factor |
| CT-1 | up | Growth factor |

Inductions labeled “up” are representing the activation from a previously undetected gene.

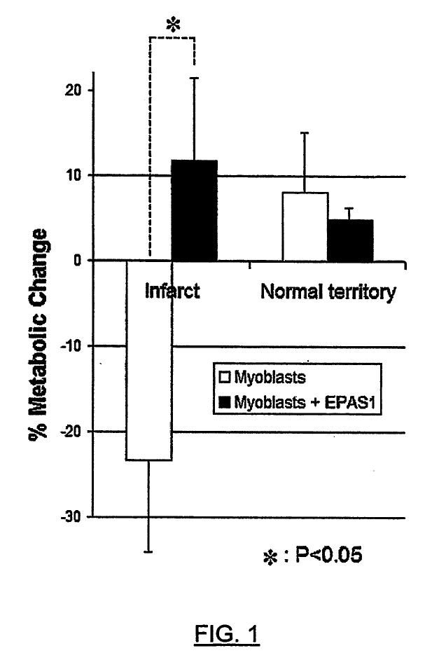

To support the idea that cell survival could be increased by EPAS1, a myoblast implantation in infarcted heart study was conducted. It was found that an improved metabolic activity was seen in infarct implanted with EPAS1 modified myoblasts, whereas a deterioration of metabolic activity was seen when unmodified myoblasts were implanted (FIG. 1). This result indicates that cell survival was improved, resulting in an increased metabolic activity.

It was shown that adrenomedullin, a cardioprotective gene, was induced by EPAS1^{(z)}, but never was it shown for cardiotrophin 1, which also have cardioprotective activity. Z: T. Tanaka et al. J Mol Cell Cardiol 2002 Endothelial PAS Domain Protein 1 (EPAS1) induces adrenomedullin gene expression in cardiac myocytes: Role of EPAS1 in an inflammatory response in cardiac myocytes. 34: 739-48.

3) Discussion

The analysis of genes activated by EPAS1 revealed the induction of several cell survival genes (Table I). These genes play a role in various aspects of cell survival and cardioprotection and the resulting improved activity is thus expected to be strong and well organized. This is a major advantage compared to the use of a single protective factor.

While several embodiments of the invention have been described, it will be understood that the present invention is capable of further modifications, and this application is intended to cover any variations, uses or adaptations of the invention, following in general the principles of the invention and including such departures from the present disclosure as to come within knowledge or customary practice in the art to which the invention pertains, and as may be applied to the essential features hereinbefore set forth and falling within the scope of the invention.

### REFERENCES

Throughout this paper, reference is made to a number of articles of scientific literature that are listed below and incorporated herein by reference:
1. 2002 Heart and stroke statistical update, American Heart Association.
2. Wang, G. L., Jiang, B.-H., Rue, E. A., and Semenza, G. L. Hypoxia-inducible factor 1 is a basic-helix-loop-helix-PAS heterodimer regulated by cellular O₂ tension. Proc. Natl. Aca. Sci. USA (1995) 92: 5510-5514.
3. Tian, H., McKnight, S. L. and Russel, D. W. Endothelial PAS domain protein 1 (EPAS1), a transcription factor selectively expressed in endothelial cells. Genes & Dev. (1996) 11: 72-82.
4. Gu, Y. Z., Moran, S. M., Hogenesch, J. B., Wartman, L. and Bradfield C A. Molecular characterization and chromosomal localization of a third alpha-class hypoxia inducible factor subunit, HIF3alpha. Gene Expression (1998). 7:205-213.
5. Jiang, B.-H., Zheng, J. Z., Leung, S. W., Roe, R. and Semenza, G. L. Transactivation and inhibitory domains of Hypoxia-inducible factor 1α. J. Biol. Chem. (1995) 272: 19253-19260.
6. Vincent, K. A., Shyu, K.-G., Luo, Y., Magner, M., Tio, R. A., Jiang, C., Goldberg, M. A., Akita, G. Y., Gregory, R. J. and Isner, J. M. Angiogenesis is induced in a rabbit model of hindlimb ischemia by naked DNA encoding an HIF-1α/VP16 hybrid transcription factor. Circulation (2000) 102: 2255-2261.
7. Staffa, A., Acheson, N. H. and Cochrane, A. Novel exonic elements that modulate splicing of the human fibronectin EDA exon. J. Biol. Chem. (1997) 272: 33394-401.
8. Tsurumi, Y., Takeshita, S., Chen, D., Kearney, M., Rossow, S. T., Passeri, J., Horowitz, J. R., Symes, J. F. and Isner J. M. Direct intramuscular gene transfer of naked DNA encoding vascular endothelial growth factor augments collateral development and tissue perfusion. Circulation. (1996) 94: 3281-3290.
9. Houle, B., Rochette-Egly, C. and Bradley, W. E. Tumor-suppressive effect of the retinoic acid receptor beta in human epidermoid lung cancer cells. Proc. Natl. Aca. Sci. USA (1993) 90: 985-989.
10. Xia et al., Cancer (2001), 91:1429-1436.
11. Isner J., Nature (2002), 415:234-239.

1. A method for increasing cell survival in cell therapy treatment, the method comprises the steps of inducing in a cell the expression of at least one cell survival gene, introducing and expressing in said cell a nucleic acid sequence encoding a functional transcription factor selected from the group consisting of EPASI, HIF-I and HIF-3 or a functional analog thereof.

2. The method of claim 1, wherein the cell survival gene is a cardioprotective gene.

3. The method of claim 2, wherein said cardioprotective gene is selected from the group consisting of LIF, LIF-R and CT-1.

4. The method of claim 3, wherein said cardioprotective gene is CT-1.

5. The method of claim 3, wherein said cardioprotective gene is LIF.

6. The method of claim 3, wherein said cardiporotective gene is LIF-R.

7. The method of claim 1, wherein said nucleic acid sequence is a cDNA.

8. The method of claim 1, wherein the cell is a mammalian cell.

9. The method of claim 8, wherein the mammalian cell is selected from the group consisting of myoblast, skeletal muscular cell, cardiomyocyte, smooth muscle cell, bone marrow cell, endothelial cell, endothelial progenitor cell, fibroblast and embryonic stem cell.

10. The method of claim 1, wherein said nucleic acid sequence is introduced into the cell using a method selected from the group consisting of adenoviral infection, and plasmid, cosmid or artificial chromosome transfection and electroporation.

11. The method of claim 10, wherein said method further comprises the step of transplanting, into the heart of a compatible recipient, a plurality of said cells.

12. The method of claim 11, wherein said transplantation is autologous.

13. The method of claim 11, wherein said transplantation improves the mammal's cardiac functions.

14. The method of claim 1, wherein said method is for the treatment of peripheral vascular disease (PVD).

15. The method of claim 1, wherein said method is for wound healing.

16. A method for increasing the metabolic activity of a muscular cell, comprising the step of introducing and expressing in said cell a nucleic acid sequence encoding a functional transcription factor of EPASI or a functional analog thereof.

17. The method of claim 16, wherein said transcription factor induces the expression of at least one cell survival gene selected from the group consisting of LIF, LIF-R, CT-1.

18. The method of claim 16, wherein said transcription factor induces the expression of a CT-1, a cardioprotective gene.

19. The method of claim 14, wherein said method is for the treatment of coronary and cardiac disorders.

20. A method for improving cardiac tissue functions of a mammal, comprising the step of providing to the cardiac tissue of said mammal a plurality of genetically modified cells expressing a nucleic acid sequence encoding a functional transcription factor of EPAS1 or a functional analog thereof.

21. The method of claim 20, wherein said genetically modified cells are provided by injecting directly said nucleotide sequence in the cardiac tissue of said mammal.

22. The method of claim 20, wherein said genetically modified cells are provided by transplanting into said cardiac tissue a plurality of cells genetically modified for expressing said transcription factor, and wherein said cells originate from a compatible donor.

23. The method of claim 22, wherein said transplantation is autologous.

24. The method of any one of claims 17 to 23, wherein said transcription factor induces the expression of at least one cell survival gene selected from the group consisting of, LIF, LIF-R, CT-1.

25. (canceled)

26. (canceled)

27. (canceled)

28. The method of claim 24, wherein the transcription factor induces the expression of CT-1 and the tissue is a muscular tissue.

29. The method of claim 24, wherein the transcription factor induces the expression of LIF and the muscular tissue is a cardiac tissue.

30. A genetically modified muscular cell expressing a functional EPAS1 transcription factor or a functional analog thereof.

31. The cell of claim 27, wherein said cell is a myoblast, a skeletal muscular cell or a cardiac cell.

32. The cell of claim 27, wherein said transcription factor is inducible.

33. The cell of claim 27, wherein said transcription factor induces the expression of at least one cell survival gene selected from the group consisting of LIF, LIF-R, CT-1.

34. The cell of claim 27, wherein said transcription factor induces the expression of CT-1.

35. The cell of 27, wherein said transcription factor induces the expression of LIF.

36. The cell of claim 27, wherein said transcription factor induces the expression of LIF-R.

37. The cell of claim 27, wherein said cell comprises a cDNA encoding said transcription factor.

38. A modified cell that contains the nucleic acid of claim 1.

39. The cell of claim 35, wherein said cell is selected from the group consisting of myoblast, mammalian skeletal muscular cells, cardiac cells, bone marrow cells, fibroblasts, smooth muscle cells, endothelial cells, endothelial progenitor cells and embryonic stem cells.

40. A transgenic animal generated from the cell of claim 35, wherein said nucleic acid is expressed in said transgenic animal.

41. (canceled)

42. (canceled)
